(19) United States
(12) Patent Application Publication
(10) Pub. No.: US 2009/0186419 A1
(43) Pub. Date: Jul. 23, 2009

(54) Luminescent Metal Oxide Films

(75) Inventors: Jackie Y. Ying, Singapore (SG); Hsiao-Hua Yu, Singapore (SG); Emril Mohamed Ali, Singapore (SG)

Correspondence Address: WOLF GREENFIELD & SACKS, P.C., 600 ATLANTIC AVENUE, BOSTON, MA, 02210-2206, US

(73) Assignee: Agency for Science ,Technology and Research, Connexis (SG)

(21) Appl. No.: 12/086,716

(22) PCT Filed: Jan. 20, 2006
(86) PCT No.: PCT/US2006/001941

§ 371 Date: Dec. 29, 2008

Related U.S. Application Data
(60) Provisional application No. 60/751,881, filed on Dec. 19, 2005.

(51) Int. Cl. G01N 33/551; B05D 5/12; G01N 33/50

(52) U.S. Cl. 436/111; 427/66; 436/129; 436/103; 436/131; 436/119; 436/98; 436/127; 436/124

(57) ABSTRACT

The present invention relates to articles and methods involving luminescent films which may be useful in various applications. Luminescent films of the present invention may comprise a layer of metal oxide nanoparticles and, in some cases, may interact with an analyte to generate a detectable signal, whereby the presence and/or amount of analyte can be determined. In some embodiments, fluorescence resonance energy transfer (FRET) may occur between the luminescent film and the analyte. Such articles and methods may be useful in, for example, biological assays or in sensors.

### FIELD OF THE INVENTION

The present invention relates to articles and methods involving luminescent films.

### BACKGROUND OF THE INVENTION

Semiconductor nanocrystals, or quantum dots, are highly emissive materials that may be useful in a variety of applications. Some semiconductor nanocrystals, such as cadmium- and lead-containing nanocrystals, have been shown to exhibit controllable emissions and narrow bandwidths, making them useful in optical devices and diagnostics, such as fluorescent probes in biological labeling. However, the broad applicability of such semiconductor nanocrystals may be limited due to their inherent toxicity. While luminescent nanoparticles having low intrinsic toxicity may be employed as an alternative, many exhibit poor photostability and limited solubility in aqueous solutions. For example, the luminescent nanoparticles may form large aggregates in aqueous solutions upon extended exposure to sunlight. Furthermore, once luminescent nanoparticles are dried and stored over an extended period of time, they may become insoluble in solution, making them incompatible for use in many applications.

Accordingly, improved methods are needed.

### SUMMARY OF THE INVENTION

The present invention provides methods for formation of a luminescent metal oxide nanoparticle thin film comprising forming a layer, comprising a luminescent metal oxide nanoparticle layer on a surface of a substrate; and heating the substrate at a temperature of no more than 150° C. for a period of time sufficient to anneal the luminescent metal oxide nanoparticle layer to the surface, wherein, prior to heating, the luminescent metal oxide nanoparticle layer has a first emission under a particular set of excitation conditions and, upon heating, the luminescent metal oxide nanoparticle layer has a second emission under the particular set of excitation conditions having at least 80% of the intensity of the first emission.

The present invention also provides methods of binding an analyte, comprising exposing a luminescent metal oxide nanoparticle layer to a sample suspected of containing an analyte and, if the analyte is present, allowing the analyte to become immobilized with respect to the luminescent metal oxide nanoparticle layer via interaction between the analyte and the luminescent metal oxide nanoparticle layer.

In another aspect, the present invention relates to articles for determination of a target analyte, comprising a substrate and a layer comprising luminescent metal oxide nanoparticles formed on and adhered to a surface of the substrate, wherein the luminescent metal oxide nanoparticles comprise a binding partner selected to preferentially bind the target analyte.

Another aspect of the present invention relates to fluorescence resonance energy transfer donors comprising a luminescent metal oxide nanoparticle comprising a binding partner selected to preferentially bind an analyte, wherein the luminescent metal oxide nanoparticle is a fluorescence resonance energy transfer donor and the analyte is a fluorescence resonance energy transfer acceptor.

### BRIEF DESCRIPTION OF THE DRAWINGS

FIG. 1 shows, schematically, the fabrication of a luminescent metal oxide layer, according to one embodiment of the invention.

FIG. 2 shows the absorption spectra of an (a) annealed and (b) unannealed ZnO nanoparticle layer after sonication in water for two minutes.

FIG. 3 shows the percentage of luminescence intensity of a ZnO nanoparticle layer after annealing at different temperatures.

FIG. 4 shows AFM images of ZnO nanoparticle films spin-coated from (a) a solution of ZnO nanoparticles in water and (b) a solution of ZnO nanoparticles in methanol.

FIG. 5 shows the kinetic luminescence measurements of (a) a ZnO nanoparticle solution and (b) a ZnO film.

FIG. 6 shows the percentage of luminescence intensity of a ZnO film in (a) the presence and (b) the absence of 1 mM o-phthaldehyde in borate buffer, and the percentage of luminescence intensity of a ZnO film in (c) the presence and (d) the absence of 1 mM o-phthaldehyde in water. The films were exposed to UV light (λₘₐₓ=365 nm) for 20 min.

FIG. 7 shows the absorption spectra (dotted line) and emission spectra (solid line) for (a) a ZnO film, excited at 345 nm; (b) a tetramethylrhodamine succinimidyl ester dye, excited at 545 nm, and (c) a ZnO film grafted with tetramethylrhodamine succinimidyl ester dye, excited at 345 nm.

FIG. 8A shows, schematically, functionalization of a ZnO layer with biotin.

FIG. 8B shows, schematically, subsequent assembly of a tetramethylrhodamine-substituted biotin/avidin/biotin-ZnO structure for fluorescence resonance energy transfer (FRET).

FIG. 9 shows, schematically, the occurrence of FRET between a luminescent ZnO layer and a tetramethylrhodamine dye bound to the ZnO layer through biotin-avidin-biotin assembly.

FIG. 10 shows the emission spectra of (a) a ZnO film grafted with biotin, excited at 345 nm, (b) a ZnO film grafted with a biotin/avidin/tetramethylrhodamine-substituted biotin assembly, excited at 345 nm, (c) a ZnO film grafted with a biotin/avidin/tetramethylrhodamine-substituted biotin assembly, excited at 545 nm, (d) an aqueous solution of tetramethylrhodamine-substituted biotin, excited at 545 nm, and (e) an aqueous solution of tetramethylrhodamine-substituted biotin, excited at 345 nm.

### DETAILED DESCRIPTION

The present invention relates to articles and methods involving luminescent films which may be useful in various applications. Luminescent films of the present invention may comprise a layer of metal oxide nanoparticles and, in some cases, may interact with an analyte to generate a detectable signal, whereby the presence and/or amount of analyte can be determined. In some embodiments, fluorescence resonance energy transfer (FRET) may occur between the luminescent film and the analyte, as discussed more fully below. Such articles and methods may be useful in, for example, biological assays or as biological sensors.

Luminescent metal oxide particles (e.g., ZnO) may be useful in, for example, biological assays and devices, due to their emissive nature and low toxicity. However, many luminescent metal oxide particles can be unstable in solution, limiting their use. For example, in aqueous solutions, some luminescent metal oxide particles form large aggregates upon extended exposure to sunlight and can be unstable at low concentrations. One approach to improving the stability of luminescent metal oxides involves incorporating them into solid-state films. However, typical previous methods for forming metal oxide films have involved calcination at high temperatures (500-700° C.), producing films that typically are significantly reduced in luminescence. The present invention provides articles and methods which, in some cases, improve the stability of luminescent metal oxide nanoparticles and apply them for use in various applications. Certain embodiments of the invention involve the fabrication and use of films comprising a layer of highly emissive, luminescent metal oxide nanoparticles, such as ZnO and other luminescent metal oxide nanoparticles, nanocrystals, or the like.

One aspect of the present invention provides methods for forming stable, luminescent metal oxide nanoparticles films (e.g., layers). In one embodiment, the method involves forming a layer comprising luminescent metal oxide nanoparticles on a surface of a substrate. The layer may be formed by deposition from a solution or suspension of luminescent metal oxide nanoparticles by, for example, spin-casting, drop-casting, or other deposition techniques, and may then be dried slowly, prior to annealing. The invention, in one aspect, involves the recognition that the drying step can affect the uniformity of the luminescent metal oxide nanoparticle layer. In some embodiments, uniform films were obtained by drying the spin-cast films at around 40° C. In some embodiments, the spin-cast films were dried slowly at room temperature. The dried films may then be heated at a temperature that is relatively mild, yet sufficient to anneal the luminescent metal oxide nanoparticle layer to the surface over an appropriate period of time. As used herein, the term “anneal” refers to the heating of a substrate and layer formed on the substrate, in order to stabilize the layer such that it adheres to the substrate, even upon immersion and/or sonication in solution. In some cases, the luminescent metal oxide nanoparticle layer forms a covalent bond to the surface upon annealing.

In some embodiments, the substrate may be heated at a temperature of no more than 150° C. during the annealing process. In other embodiments, the film is heated at a temperature of no more than 140° C., no more than 130° C., no more than 120° C., or no more than 110° C. Also, the film may be heated for a period of time sufficient to form a stable (e.g., annealed) layer to the surface of the substrate without diminishing the optical properties of the layer. In one embodiment, the film may be annealed for about ten minutes. Both the temperature and the duration of annealing step may influence the properties of the resulting film, such as film uniformity and luminescence (e.g., fluorescence, phosphorescence, and the like). Those of ordinary skill in the art, with the benefit of this disclosure, will be able to select combinations of annealing temperatures and times, in conjunction with metal oxides, to produce good films without any or much loss in luminescence of metal oxide without undue experimentation.

Preferred luminescent metal oxide nanoparticle films of the invention are robust and photostable upon annealing. In some embodiments, the luminescent metal oxide nanoparticle layer substantially retains its luminescent properties upon annealing. For example, prior to exposure to annealing conditions of the invention (heating at a temperature and for a period of time sufficient to anneal the film relative to the substrate), a luminescent metal oxide nanoparticle layer may have a first emission under a particular set of excitation conditions. After exposure to such conditions, the luminescent metal oxide nanoparticle layer may have a second emission under the same particular set of excitation conditions, wherein the second emission has at least 80% of the intensity of the first emission at at least one emissive wavelength. An “emissive wavelength” means a wavelength at which the subject material emits both before and after annealing, and one that can serve a useful signaling function in an assay or the like. In another embodiment, the second emission has at least 90% of the intensity of the first emission. The retention of a substantial majority of the luminescence properties of the film may be attributed to the relatively low annealing temperature. As shown in FIG. 3, the luminescence intensity of an annealed film in one comparative study of the invention decreases as the annealing temperature is increased. When a film is heated to 500° C., more than 98% of the luminescence was diminished in this example. In some embodiments, luminescent metal oxide nanoparticle layers of the invention may be annealed at a temperature of no more than 110° C. in order to preserve at least 90% of the luminescence intensity of the pre-annealed e.g., spin-cast) layer.

In some embodiments, the luminescent metal oxide films or layers have significant uniformity. That is, the luminescent metal oxide nanoparticles may be evenly distributed within the layer and across the surface of the substrate, rather than forming aggregates. Also, the films may be resistant to dissolution upon annealing, in some cases, due to formation of covalent bonds between, for example, hydroxy groups on the metal oxide nanoparticles and surface groups (e.g., silanol groups) of the substrate (e.g., glass substrate). In some cases, the luminescent metal oxide nanoparticle layers can be stored for extended periods of time (e.g., one week, one month, three months, or even 6 months or a year), at room temperature (about 25° C.) and/or near room temperatures (i.e., between about 4° C. and about 25° C.), without significant deterioration (e.g., with less than 1%, 2%, 5%, 10%, 15%, or 20% loss) of luminescence at at least one emissive wavelength. Annealed films of the invention, in other embodiments, are stable as noted above even if stored at temperatures of at least 30° C., 35° C., 40° C., or 45° C.

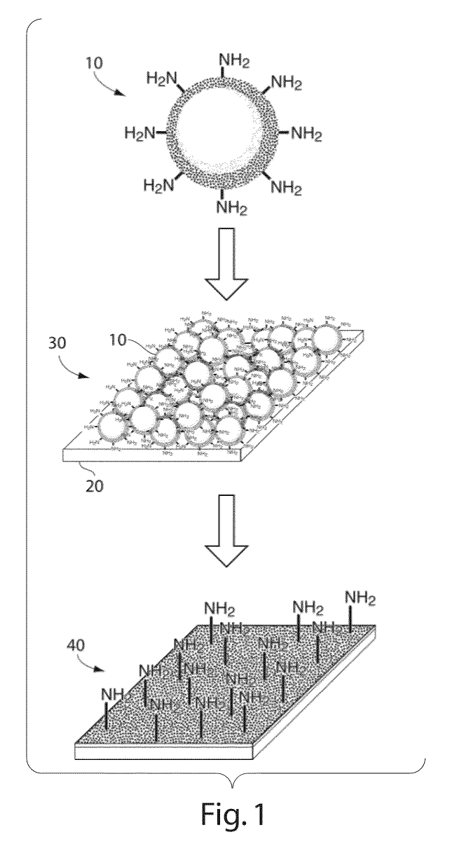

In an illustrative embodiment shown in FIG. 1, a luminescent ZnO film may be prepared from a solution of ZnO nanoparticles. A solution (e.g., aqueous solution) of luminescent particle 10, which comprises a silane coating functionalized with amine groups at the surface, may be deposited (e.g., spin-cast, drop-cast, or the like) on a substrate 20 to form article 30, dried at 40° C., and then annealed at 110° C. to form the ZnO film 40. The ZnO films are highly uniform, robust and photostable. The ZnO films show better photostability and similar reactivity, compared to the corresponding luminescent ZnO nanoparticles solution.

The present invention also provides articles comprising a substrate and a luminescent metal oxide nanoparticle layers formed on and adhered to a surface of the substrate, wherein the luminescent metal oxide nanoparticles comprise a plurality of functional groups. In some cases, the functional group may be presented at and may confer a specific property to the surface of the luminescent metal oxide nanoparticle layer. That is, the functional group may include a functionality that, when presented at the surface of the layer, may be able to confer upon the surface a specific property, such as an affinity for a particular entity or entities. In some embodiments, the functional group may act as a binding partner and may form a bond (e.g., a covalent, ionic, hydrogen, or dative bond, or the like) with an analyte. Those of ordinary skill in the art, with the benefit of this disclosure, will be able to select such functional groups without undue experimentation. Examples of suitable functional groups include, but are not limited to, —OH, —CONH—, —CONHCO—, —NH₂, —NH—, —COOH, —COOR, —CSNH—, —NO₂⁻—, —SO₂⁻—, —RCOR—, —RCSR—, —RSR, —ROR—, —PO₄⁻³, —OSO₃⁻², —COO—, —SOO⁻, —RSOR—, —CONR₂, —CH₃, —PO₃H⁻, -2-imidazole, —N(CH₃)₂, —NR₂, —PO₃H₂, —CN, —(CF₂)ₙ—CF₃ (where n=1-20 inclusive, and preferably 1-8, 3-6, or 4-5), olefins, and the like. In some embodiments, the binding partner may be selected from among amine, carboxylic acid, phosphate, hydroxyl, and thiol. In certain embodiments, the luminescent metal oxide nanoparticle layer comprise amines presented at its surface, and in other embodiments, the luminescent metal oxide nanoparticle layer comprise carboxylic acids presented at its surface.

In some embodiments, the functional group may be further functionalized with a binding partner selected to preferentially bind a target analyte by, for example, binding between two biological molecules or formation of a bond. The binding partner may be a chelating group, an affinity tag (e.g., a member of a biotin/avidin or biotin/streptavidin binding pair or the like), an antibody, a peptide or protein sequence, a nucleic acid sequence, or a moiety that selectively binds various biological, biochemical, or other chemical species. In one embodiment, the binding partner may comprise avidin.

Another aspect of the invention relates to methods for binding an analyte. Luminescent metal oxide nanoparticle layers of the invention may be exposed to a sample suspected of containing an analyte and, if the analyte is present, the analyte may become immobilized with respect to the luminescent metal oxide nanoparticle layer via interaction between the analyte and the luminescent metal oxide nanoparticle layer. As described herein, the analyte may interact with the luminescent metal oxide nanoparticle layer via binding between two biological molecules or, in some cases, via formation of a bond.

As used herein, “binding” can involve any hydrophobic, non-specific, or specific interaction, and “binding between two biological molecules” refers to the interaction between a corresponding pair of molecules that exhibit mutual affinity or binding capacity, typically specific or non-specific binding or interaction. The interaction of the luminescent metal oxide nanoparticle layer and the fluorophore, in some instances, may be facilitated through specific interactions, such as a protein/carbohydrate interaction, a ligand/receptor interaction, or other biological binding partners. The term “binding partner” refers to a molecule that can undergo binding with a particular molecule. The term “specific interaction” is given its ordinary meaning as used in the art, i.e., an interaction between pairs of molecules where the molecules have a higher recognition or affinity for each other than for other, dissimilar molecules. Biotin/avidin and biotin/streptavidin are examples of specific interactions.

The ability of luminescent metal oxide nanoparticle layers to preferentially bind analytes may advantageous for a number of applications. For example, in one embodiment, the present invention provides methods for fluorescence resonance energy transfer (FRET) between the luminescent metal oxide nanoparticle and a fluorophore. The term “fluorescence resonance energy transfer” or “FRET” is known in the art and refers to the transfer of excitation energy from an excited state species (i.e., FRET donor) to an acceptor species (i.e., FRET acceptor), wherein an emission is observed from the acceptor species. The luminescent metal oxide nanoparticle layer may interact such that FRET may occur. The interaction may comprise interaction of the luminescent metal oxide nanoparticle layer with an analyte, wherein the analyte is a fluorophore. In some cases, the analyte may comprise a fluorophore. For example, the analyte may be linked to the fluorophore via a bond or a binding interaction, or may be otherwise associated with the fluorophore. As used herein, the term “analyte” should be understood to comprise a fluorophore associated with the analyte.

The present invention may provide methods wherein the articles described herein may undergo FRET with an analyte, such that the analyte facilitates energy transfer between an energy donor and an energy acceptor. For example, the analyte comprising a fluorophore (the analyte can, itself, be a fluorophore and/or the analyte can be attached or otherwise immobilized with respect to a fluorophore) may be exposed to a luminescent metal oxide nanoparticle layer, wherein the luminescent metal oxide layer is a FRET donor and the fluorophore is a FRET acceptor. The analyte may become immobilized with respect to the luminescent metal oxide nanoparticle layer, such that the fluorophore is positioned in sufficient proximity to the luminescent metal oxide nanoparticle layer to enable the occurrence of FRET, as would be understood by those of ordinary skill in the art. Exposure of the luminescent metal oxide layer to a source of energy may form a luminescent metal oxide layer excitation energy, which may then be transferred to the fluorophore, causing an emission from the fluorophore. The analyte may be determined (e.g., observed, quantified, etc.) by the emission. Such methods may allow for reduced photobleaching of fluorophores, since the fluorophores may not undergo direct excitation by electromagnetic radiation, which may prolong and/or improve the performance of fluorophores, such as small organic molecules, fluorescent dyes, green fluorescent proteins, and the like. In some cases, FRET may result in an amplification of emission of a fluorophore, allowing for more reliable quantification of fluorescence emission. Also, methods of the invention may be advantageous in systems where the fluorophore concentration may be low.

The analyte and the luminescent metal oxide nanoparticle layer may be brought in proximity to each other using specific interactions, such that the luminescent metal oxide nanoparticle layer (e.g., the energy donor) and a fluorophore associated with an analyte (e.g., the energy acceptor) can participate in energy transfer. For example, the luminescent metal oxide nanoparticle layer may comprise a ligand and the analyte may comprise a receptor to that ligand. In one embodiment, the luminescent metal oxide nanoparticle layer comprises biotin and the analyte may comprise avidin or streptavidin. Alternatively, the luminescent metal oxide nanoparticle layer may comprise a biotin-avidin complex and the analyte may comprise biotin. In another example, the luminescent metal oxide nanoparticle layer may comprise an oligonucleotide (DNA and/or RNA) and the analyte may comprise a substantially complementary oligonucleotide. Those of ordinary skill in the art would be able to select the appropriate pair of binding partners that would suit a particular application.

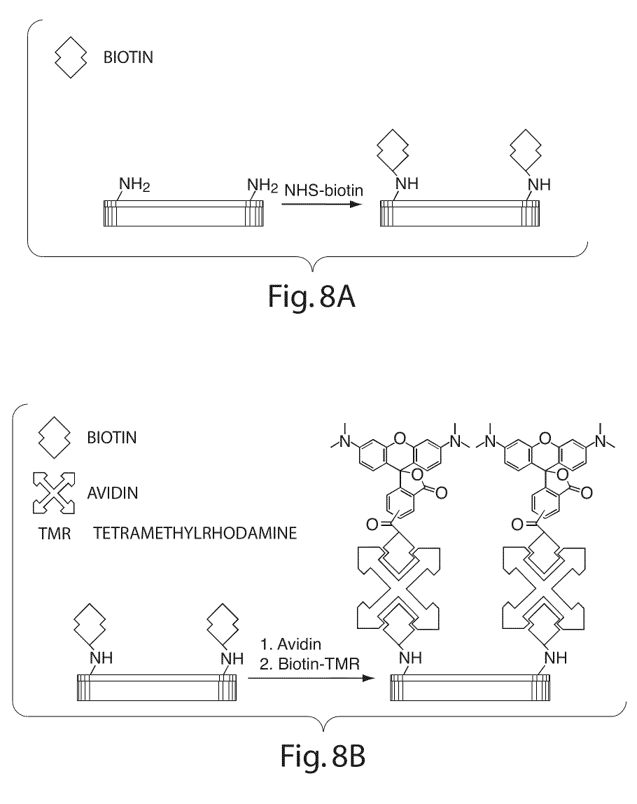

In some embodiments, an intermediate binder may facilitate bringing the luminescent metal oxide nanoparticle layer and the analyte into sufficient proximity with one another to facilitate FRET. For example, the intermediate binder may specifically bind to the luminescent metal oxide nanoparticle layer and to the analyte. The intermediate binder, the luminescent metal oxide nanoparticle layer, and the analyte may interact in any order, so long as the chromophores are brought into proximity with each other. For example, the luminescent metal oxide nanoparticle layer and the intermediate binder may first interact, then the analyte may interact with one or both of the luminescent metal oxide nanoparticle layer and the intermediate binder; the luminescent metal oxide nanoparticle layer and the analyte may first interact, then one or both of the luminescent metal oxide nanoparticle layer and the analyte may interact with an analyte; the analyte, the luminescent metal oxide nanoparticle layer, and the analyte may all simultaneously interact; or the like. In a particular embodiment, the luminescent metal oxide nanoparticle layer and the analyte each comprise biotin, and the intermediate binder comprises avidin, as shown schematically in FIG. 8. Interaction of the luminescent metal oxide nanoparticle layer and/or the analyte with the intermediate binder may give an emission having a threshold level that, in the absence of the intermediate binder, the luminescent metal oxide nanoparticle layer and/or the analyte do not produce an emission that is at or above the emission threshold level.

In another aspect, the present invention provides a FRET donor comprising luminescent metal oxide nanoparticles comprising a binding partner selected to preferentially bind an analyte, wherein FRET may occur between the luminescent metal oxide nanoparticles and the analyte, as described herein. Application of electromagnetic energy at the excitation wavelength of the of the luminescent metal oxide nanoparticle layer may generate a luminescent metal oxide nanoparticle excitation energy, which may then be transferred to the fluorophore, causing an emission from the fluorophore. The luminescent metal oxide nanoparticle and the fluorophore may be selected to facilitate efficient FRET. For example, the luminescent metal oxide nanoparticle may have an emission spectrum that overlaps with the absorption spectrum of the fluorophore.

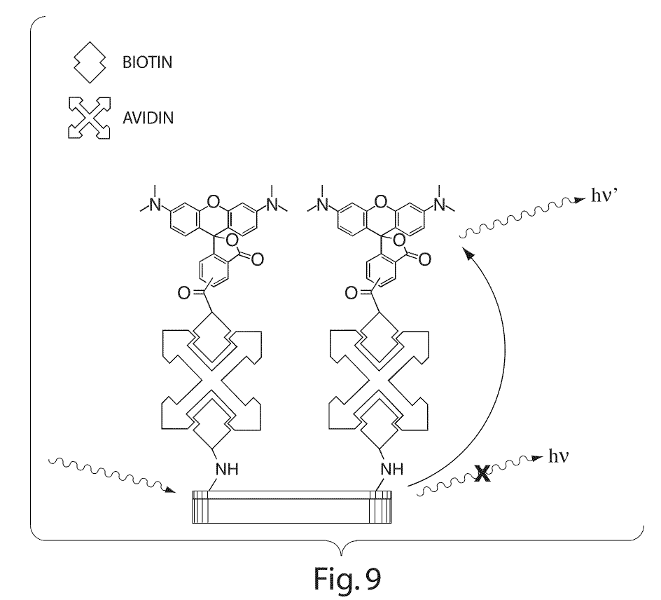

In some cases, the fluorophore may be an organic, fluorescent dye, wherein the excitation at the wavelength of the luminescent metal oxide nanoparticle layer causes FRET from the layer to the dye, resulting in a emission peak from the dye. FIG. 9 shows, schematically, an illustrative embodiment of the invention, wherein excitation of the luminescent metal oxide nanoparticle layer results in an emission peak from the bound rhodamine dye. Examples of fluorescent dyes include, but are not limited to, fluorescein, rhodamine B, Texas Red™ X, sulforhodamine, calcein, and the like.

The use of luminescent metal oxide nanoparticle layers as FRET donors may be advantageous in several applications, as the layer may act as an effective light-harvesting tool for generating a detectable signal. As a result, devices (e.g, sensors) and assays incorporating articles and methods of the invention may be highly sensitive and selective for a given analyte. For example, the emission intensity of an organic dye due to FRET from a luminescent metal oxide nanoparticle layer may be substantially higher than the emission intensity of the same organic dye due to direct excitation of the organic dye. This may be advantageous in, for example, systems having low concentrations of analyte. The amplification of emission intensity due to FRET from the luminescent metal oxide nanoparticle layer may be particularly useful in the determination of analytes in bioassays, fluorescent labeling of biomolecules, sensing and quantification of biomolecules and other chemicals, and the like.

The luminescent metal oxide nanoparticle layer may also be useful for devices (e.g., sensors) and methods for determining analytes, such as chemical or biological analytes, wherein the analyte may become immobilized with respect to the luminescent metal oxide layer and FRET may occur between the layer and the analyte, as described herein. As used herein, the term “determining” generally refers to the analysis of a species or signal, for example, quantitatively or qualitatively, and/or the detection of the presence or absence of the species or signals. “Determining” may also refer to the analysis of an interaction between two or more species or signals, for example, quantitatively or qualitatively, and/or by detecting the presence or absence of the interaction. The present invention may provide articles and methods for determining a biological entity in a sample, for example, determining the presence, type, amount, etc. of the biological entity within a sample. The sample may be taken from any suitable source where the presence of the biological entity is to be determined, for example, from food, water, plants, animals, bodily fluids (for example lymph, saliva, blood, urine, milk and breast secretions, etc.), tissue samples, environmental samples (for example, air, water, soil, plants, animals, etc.), or the like. In one embodiment, the biological entity is a pathogen.

For example, the present invention provides, in one embodiment, a method that involves exposing a luminescent metal oxide nanoparticle layer to a sample suspected of containing an analyte comprising a fluorophore, wherein the luminescent metal oxide nanoparticle layer is an energy donor and the fluorophore is an energy acceptor, as described herein. In the event that the analyte is present, the analyte can be determined via determination of an emission from the fluorophore, as described herein.

FIG. 8 shows an illustrative embodiment wherein a luminescent metal oxide nanoparticle layer comprises a biotin binding partner presented at the surface of the layer (FIG. 8A). Exposure of the luminescent metal oxide nanoparticle layer to avidin and a fluorescent-tagged biotin causes the avidin and fluorescent-tagged biotin to bind to the layer via interaction between the avidin and biotin moieties (FIG. 8B). As shown in FIG. 9, application of electromagnetic energy at the excitation wavelength of the of the luminescent metal oxide nanoparticle layer may generate a luminescent metal oxide nanoparticle excitation energy, which may then be transferred to the fluorescent tag, causing substantial portion of the emission to occur from the fluorescent tag, rather than from the luminescent metal oxide nanoparticle layer. The occurrence of this emission may indicate the presence and/or amount of analyte present in the sample.

Various embodiments of the invention provide for the transfer of energy from an energy donor to an energy acceptor. In some embodiments, the luminescent metal oxide nanoparticle layer may be the energy donor and an fluorophore may be the energy acceptor. Alternatively, in other embodiments, the luminescent metal oxide nanoparticle layer may be selected to be the energy donor and a fluorophore may be the energy acceptor. Those of ordinary skill in the art would be able to select the appropriate materials for use as energy donors and/or acceptors.

For example, the energy acceptor or donor in a FRET mechanism may be chosen based on the wavelength of absorbance and/or emission. Energy may be transferred from the an energy donor to the energy acceptor through Förster transfer, a Dexter mechanism, or a combination of Förster transfer and a Dexter mechanism. In cases where Förster transfer is the mechanism of energy transfer between the energy donor and acceptor, the degree of energy transfer may vary with the amount of spectral overlap between the energy donor emission and the energy acceptor absorbance. In cases where the energy transfer can occur by a Dexter mechanism, the amount of energy transfer may be substantially independent of the spectral overlap between the energy donor and acceptor. As used herein, “spectral overlap” is given its ordinary meaning as used in the art, i.e., when two spectra are normalized and superimposed, an area exists that is simultaneously under both curves (i.e., as determined by integrals).

In another set of embodiments involving FRET, the first chromophore (e.g., the energy donor) may have a first emission lifetime and the second chromophore (e.g., the energy acceptor) may have a second emission lifetime at least about 5 times greater than the first emission lifetime, and in some cases, at least about 10 times greater, at least about 15 times greater, at least about 20 times greater, at least about 25 times greater, at least about 35 times greater, at least about 50 times greater, at least about 75 times greater, at least about 100 times greater, at least about 125 times greater, at least about 150 times greater, at least about 200 times greater, at least about 250 times greater, at least about 350 times greater, at least about 500 times greater, etc.

In yet another set of embodiments, the second chromophore may enhance emission of the first chromophore, for example, by a factor of at least about 5-fold, at least about 10-fold, at least about 30-fold, at least about 100-fold, at least about 300-fold, at least about 1000-fold, at least about 3000-fold, or at least about 10,000-fold or more in some cases.

In some cases, FRET may give rise to new threshold emissions in the presence of the analyte, where the new threshold emissions have minimal overlap with emissions in the absence of analyte. In one set of embodiments, the new threshold emission may have a peak maximum of at least about 100 nm higher in wavelength than that of the dominant non-threshold emission, i.e., the energy donor and the energy acceptor may have maximum emission wavelengths that differ by at least about 100 nm. In other cases, the new threshold emission may have a peak maximum of at least about 150 nm higher in wavelength than that of the dominant non-threshold emission. In yet other cases, the new threshold emission may have a peak maximum of at least about 200 nm, about 250 nm, about 300 nm, or more higher in wavelength than that of the dominant non-threshold emission.

The luminescent metal oxide nanoparticle layers may be formed by any suitable method known to those of ordinary skill in the art, including solvent casting techniques such as spin-casting, drop-casting or slow evaporation. The temperature and duration of the annealing step may be varied to suit a particular application. In some embodiments, the annealing temperature and duration may be varied to optimize certain properties, such as adhesion to the substrate and the optical properties of the layer. For example, the temperature and time may be selected to be sufficient to adhere the layer to the surface of the substrate, in some cases, by formation of a covalent bond. This may be evaluated by testing by immersing and/or sonicating the annealed layer in solution to determine if the layer remains adhered to or becomes detached from the substrate. Similarly, the luminescence of the layer may be observed at several temperatures and/or time intervals to determine if, and at what temperature and/or time period, the optical properties may begin to diminish.

Functional groups and/or binding partners may be may be attached to luminescent metal oxide nanoparticles using known methods. In order to functionalize the surface of the luminescent metal oxide nanoparticles, the nanoparticles may, in some cases, first be reacted with a functionalized silane in the presence of a controlled amount of base such that the functionalized silane undergoes substantially only a single hydrolysis reaction, forming a covalent bond with the nanoparticle. The degree and rate of silane conjugation can be controlled by varying the temperature and the amount of base in the reaction system. The intermediate isolated from the first step may then be suspended in a solvent where it is then reacted with an excess of a base to complete the intraparticle silanization of the functionalized silane moieties.

Silane conjugation may be carried out with various types of silanes, including those having trimethoxy silyl, methoxy silyl, or silanol groups at one end, which may be hydrolyzed in basic medium to form a silica shell around the nanoparticle. The silanes may also comprise organic functional groups, examples of which include phosphate and phosphonate groups, amine groups, thiol groups, carbonyl groups (e.g., carboxylic acids, and the like), C₁-C₂₀ alkyl, C₁-C₂₀ alkene, C₁-C₂₀ alkyne, azido groups, epoxy groups, or other functional groups described herein. These functional groups may be bound to the functionalized silanes prior to or subsequent to silane conjugation to the nanoparticle, using methods known in the art. Also, the functional groups may be presented at the surface of the luminescent metal oxide nanoparticles and luminescent metal oxide nanoparticle layers.

As described herein, the luminescent metal oxide nanoparticles may also comprise a binding partner selected to preferentially bind a target analyte. The binding partner may comprise a biological or a chemical molecule able to bind to another biological or chemical molecule in a medium, e.g. in solution. For example, the binding partner may be capable of biologically binding an analyte via an interaction that occurs between pairs of biological molecules including proteins, nucleic acids, glycoproteins, carbohydrates, hormones, and the like. Specific examples include an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair.

Luminescent metal oxide nanoparticles of the invention may be synthesized using methods known in the art, including methods described in Jana et al., Chem. Mater. 2004, 16, 3931-3935; Meulenkamp et al., J. Phys. Chem. B 1998, 102, 5566; Abdullah et al., Adv. Func. Mater. 2003, 13, 800, each incorporated herein by reference. The term “nanoparticle” may refer a particle having a maximum cross-sectional dimension of no more than 1 μm. Nanoparticles can be made of material that is, e.g., inorganic or organic, polymeric, ceramic, semiconductor, metallic, non-metallic, crystalline (e.g., “nanocrystals”, amorphous, or a combination. Typically, nanoparticles are of less than 250 nm cross section in any dimension, more typically less than 100 nm cross section in any dimension, and preferably less than 50 nm cross section in any dimension. In some embodiments, the nanoparticles may have a diameter of about 2 to about 50 nm. In some embodiments, the nanoparticles may have a diameter of about 2 to about 20 nm. In further embodiments, the nanoparticles may have diameters of about 2 to about 3 nanometers.

Metal oxides that may be used in the present invention may be an oxide of a Group 1-17 metal. Examples of suitable metal oxide nanoparticles include, but are not limited to, zinc oxide, iron oxide, manganese oxide, nickel oxide, and chromium oxide. In a particular embodiment, the luminescent metal oxide nanoparticle comprises ZnO nanoparticles. Those of ordinary skill in the art would be able to select the appropriate metal oxide to suit a particular application.

Various screening tests may be employed to determine the appropriate choice of metal oxide for use in the present invention. For example, in some cases, the metal oxide may be chosen based on the ability of the metal oxide to adhere to a substrate, such as a glass substrate. In some cases, a layer of metal oxide nanoparticles comprising free hydroxyl groups at the surface may be capable of adhering to a glass substrate via formation of covalent bonds between the layer and the substrate. In some cases, the metal oxide may be chosen such that nanoparticles of the metal oxide may be annealed and adhered to a substrate at relatively mild temperatures (e.g., no more than 150° C.). One screening test may involve forming a layer of metal oxide nanoparticles on a substrate and annealing the substrate as described herein. The annealed film may then be immersed and/or sonicated in solution to determine if the film remains adhered to or becomes detached from the substrate.

Another screening test may involve the evaluation of the ability of the metal oxide exhibit luminescence and to substantially retain the luminescence upon annealing. A layer of metal oxide nanoparticles may be formed on a substrate, and its optical properties (e.g., absorbance, emission, etc.) may be measured. Upon annealing, the optical properties of the layer may be measured and compared to the optical properties measure prior to annealing. In some cases, metal oxide nanoparticles that retain at least 80% or at least 90% of their luminescence upon annealing may be desirable for use in the present invention.

It may be also desirable to utilize a substantially non-toxic metal oxide, such as ZnO, for certain applications (e.g., applications related to biological molecules). The metal oxide may be chosen based on the ability to interact with biomolecules, such as cells, proteins, and the like, with either no or minimal disruption and/or damage to the biomolecule. A simple screening test may involve the addition of metal oxide nanoparticle to a sample containing a biomolecule (e.g., cell culture media, and the like) and observing the response of the biomolecule to the metal oxide nanoparticle.

In one set of embodiments, the luminescent metal oxide nanoparticle layer may interact with an analyte, i.e., a molecule or other moiety that is able to emit radiation upon interacting with the luminescent metal oxide nanoparticle layer. The term “analyte,” may refer to any chemical, biochemical, or biological entity (e.g. a molecule) to be analyzed. In some cases, luminescent metal oxide nanoparticle layers of the present invention may have high specificity for the analyte, and may be a chemical, biological, or explosives sensor, for example. The analyte may be or may comprise a cluomophore or a fluorophore. For example, the analyte may be a commercially available analyte, for example, but not limited to, fluorescein, rhodamine B, Texas Red™X, sulforhodamine, calcein, etc. In certain embodiments, the analyte itself may comprise a luminescent metal oxide nanoparticle layer. In some cases, interaction between the analyte and the luminescent metal oxide nanoparticle layer may be facilitated by an intermediate linker, as described herein. In some cases, the interaction between the analyte and the luminescent metal oxide nanoparticle layer may also alter the emission of the luminescent metal oxide nanoparticle layer. In some cases, the luminescent metal oxide nanoparticle layer and the analyte may interact through an energy exchange mechanism, such as a Dexter or Förster energy transfer mechanism.

In some cases, the analyte may be chosen such that the emission of the analyte does not have a high degree of spectral overlap with the emission of the luminescent metal oxide nanoparticle layer, as further discussed below. Thus, the analyte may be chosen to reduce stray light (background) emissions, which may lead to increased sensitivity and more sensitive sensors in various embodiments of the invention.

In some cases, an analyte may become immobilized with respect to articles of the present invention. As used herein, a component that is “immobilized with respect to”another component either is fastened to the other component or is indirectly fastened to the other component, e.g., by being fastened to a third component to which the other component also is fastened, or otherwise is translationally associated with the other component. For example, an analyte is immobilized with respect to a luminescent metal oxide nanoparticle layer if analyte is fastened to a binding partner attached to the layer, is fastened to an intermediate binder to which the binding partner attached to the layer is fastened, etc. In some embodiments, the analyte comprises a moiety that is capable of interacting with at least a portion of the luminescent metal oxide nanoparticle layer. For example, the moiety may interact with the layer by forming a bond, such as a covalent bond, or by binding (e.g., biological binding) as described herein.

### EXAMPLES

General Procedures. All chemicals were purchased from commercial sources (Alfa Aesar, Gelest, Lancaster and Sigma-Aldrich) unless otherwise specified, and were used without further purification. Tetramethylrhodamine dye and its derivatives were purchased from Invitrogen. Absorption spectra of samples were obtained at room temperature with an Agilent 8453 UV-Vis spectrometer. Luminescence spectra were measured at room temperature on a Jobin Yvon Horiba Fluorolog luminescence spectrometer. AFM micrographs were taken with a Vecco Multimode atomic force microscope. Spin-coating was carried out on Laurell WS-400B-6NPP-LITE spin coater.

### Example 1

As illustrated in FIG. 1, a luminescent metal oxide nanoparticle layer was formed by spin-casting an aqueous solution of amine-terminated metal oxide nanoparticles onto a glass substrate and annealing to form a luminescent film.

Immediately prior to use, amine-functionalized ZnO (NH₂—ZnO) nanoparticles (˜30 mg) were dissolved in 10 mL of deionized water and filtered through a 0.21 μm membrane syringe filter. The concentration of NH₂—ZnO nanoparticles solution was quaritified by UV-visible spectrometry at a wavelength of 330 nm. Pyrex glass substrates were diced to the desired size by a Disco DAD3350 automatic dicing saw, and cleaned by sonication in NaOH solution, HCl solution, and deionized water, sequentially, prior to use. Low spinning speeds were used due to the high viscosity of the stock solution. For a 2 cm×2 cm substrate, 680 μL of the stock NH₂—ZnO solution (0.3 mg/mL) was dropped onto the glass surface, which was spun at 240 rpm for 10 min. For a 1 cm×1 cm substrate, 120 μL of the stock NH₂—ZnO solution (0.3 mg/mL) was dropped onto the glass surface, which was spun at 500 rpm for 10 min.

Uniform films were obtained when the coatings were dried slowly at 40° C. until all solvents were evaporated. When the films were dried at room temperature, they displayed a circular pattern. Drying at temperatures above 40° C. led to films with reduced luminescence intensity, possibly due to lattice distortion. However, the NH₂—ZnO film could be redissolved by sonication in water for 2 min. In order to increase the adhesion between the NH₂—ZnO film and the glass substrate, the coated substrate was annealed at 110° C. for 10 min. FIG. 1B shows that the NH₂—ZnO film became resistant to dissolution after annealing, potentially due to covalent bond formation between the uncapped —OH groups of the ZnO nanoparticles and the surface silanol groups of the glass substrate.

### Example 2

In this comparative example, the mildness of the annealing temperature was shown to be important in the preparation of uniformly emissive films. As shown in FIG. 3, more than 98% luminescence was lost (e.g., quenched) when the films were heated to 500° C. When the annealing temperature was lowered to 110° C., at least 90% of the luminescence intensity of the films were retained. Other details were identical or similar to those described in Example 1.

### Example 3

The surface functionalization, uniformity, optical properties, and stability of the luminescent metal oxide nanoparticle films fabricated as described in Example 1 were evaluated.

Addition of fluorescamine, which reacts rapidly with primaly amine groups, to the NH₂—ZnO film showed binding of the fluorescamine to the film, indicated that NH₂ groups were presented at the surface of the film.

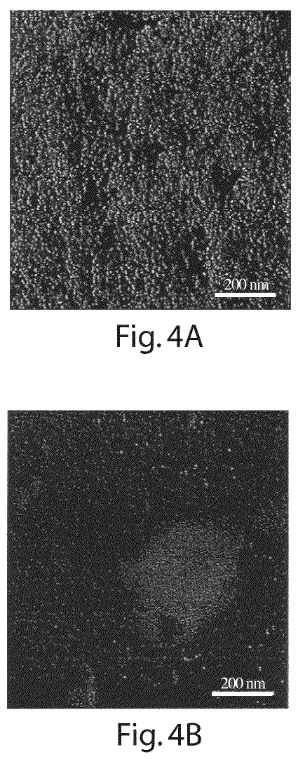

Atomic force microscopy (AFM) images were obtained of the films. As shown in FIG. 4A, the films spin-coated NH₂—ZnO from aqueous (e.g., water) solution have good uniformity. In contrast, films spin-coated from NH₂—ZnO nanoparticles solution in methanol produced aggregates of nanoparticles on the glass surface, which has a NH₂—ZnO coverage of less than 25% (FIG. 4B).

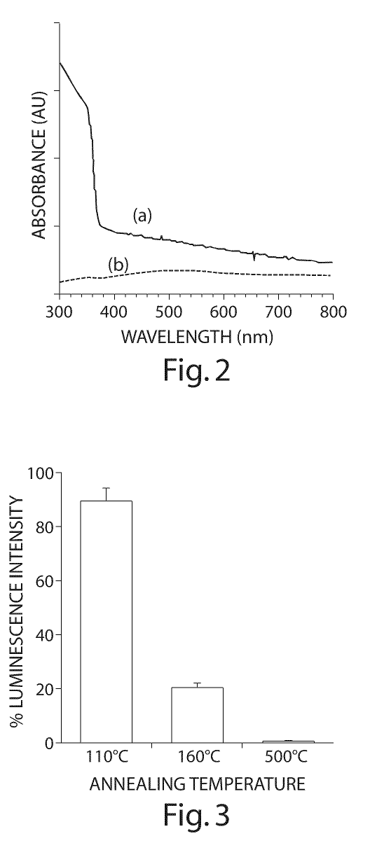

FIG. 2 shows the absorption spectra of an (a) annealed and (b) unannealed ZnO nanoparticle layer after sonication in water for two minutes. Similar to NH₂—ZnO nanoparticles in solution, the annealed NH₂—ZnO film showed a broad absorption in the UV region, which decreased sharply above 350 nm (FIG. 2A). The emission peak of the films shifted slightly to 537 nm, in contrast to 545 nm for the solution. The unannealed ZnO nanoparticle layer showed substantially no absorbance spectrum upon sonication, indicating that the nanoparticles were no longer adhered to the surface of the substrate (FIG. 2B).

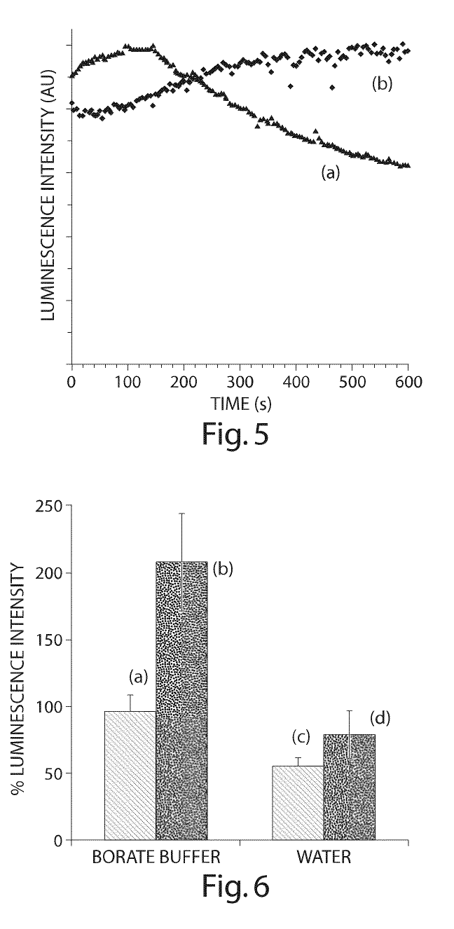

In solution, the NH₂—ZnO nanoparticles were observed to form aggregates after extended exposure to sunlight, and the luminescence intensity of the solution showed a 20% reduction upon exposure to UV light (FIG. 5A). In contrast, NH₂—ZnO films displayed robustness upon UV irradiation. The luminescence intensity at 545 nm increased by at least 20% when the film was continuously excited at 345 nm for 10 min (FIG. 5B). The increase in luminescence may be due to the lattice perfection of ZnO under continuous irradiation. At least 60% of luminescence of the original film was preserved after the NH₂—ZnO film had been stored in open air at 4° C. for three months.

### Example 4

The ability of analytes to affect certain luminescence characteristics of the NH₂—ZnO nanoparticle layer fabricated as described in Example 1 was evaluated. Luminescent metal oxide nanoparticles of the invention may comprise a luminescent core (e.g., ZnO) and a protective outer layer (e.g., silane layer), which may be a tightly-packed structure at the surface of the nanoparticle. The outer layer may comprises alkyl or heteroalkyl chains having terminal amine groups, which may react with aldehydes reversibly to form imines. The presence of the outer layer may provide chemical and photochemical stability to the luminescent core upon, for example, exposure to electromagnetic radiation (e.g., UV light). Exposure of a luminescent metal oxide nanoparticle layer to an aldehyde-substituted analyte may result in the formation of a covalent bond between luminescent metal oxide nanoparticle layer and the aldehyde-substituted analyte via imine formation, causing the outer layer to become dispersed from the surface of the nanoparticle. That is, the chains may become elongated such that the imine moiety is increased in separation from the surface. In some cases, this may be due to a change in affinity of the outer layer for the surface of the nanoparticle. In some cases, the outer layer may become dispersed, for example, by the elongation of alkyl or heteroalkyl chains due to the size of the analyte bonded to the outer layer. For example, the analyte may be a sterically bulky analyte, such as a protein or other biological analyte, which may prevent formation of a tightly-packed outer layer. The dissolution of the tightly-packed structure of the outer layer may result in the loss of photostability and occurrence of photobleaching upon exposure to electromagnetic radiation (e.g., UV, visible, IR, etc.), indicating the presence or amount of the analyte.

In one example, the NH₂—ZnO films were exposed to o-phthaldehyde in either borate buffer or in water (FIG. 6). The NH₂—ZnO films were placed in 6- or 24-well plates. Upon addition of 1 mM o-phthaldehyde solution in water or 10 mM borate buffer to each well, the plate was exposed to UV light (λₘₐₓ=365 nm, 50 W) from a flat-panel transilluminator (Wealtec) for 2 min and the emission spectra were obtained.

In the absence of aldehyde-substituted analyte, the luminescence intensity of the NH₂—ZnO film was observed to be significantly higher in borate buffer than in water (FIG. 6B and FIG. 6D, respectively), indicating that the presence of the buffer stabilized the NH₂—ZnO nanoparticles. In the presence of o-phthaldehyde, the luminescence intensity of the NH₂—ZnO films was decreased upon UV irradiation for 2 min. FIG. 6 shows the percentage of luminescence intensity of a ZnO film in the presence (FIG. 6A) and the absence (FIG. 6B) of 1 mM o-phthaldehyde in borate buffer. The luminescence intensity of the film decreased by approximately 50% in the presence of aldehyde. The percentage of luminescence intensity of a ZnO film in the presence (FIG. 6C) and the absence (FIG. 6D) of 1 mM o-phthaldehyde in water was also measured. The luminescence intensity of the film decreased slightly in the presence of aldehyde. This may be due to reaction of the aldehyde with the surface amine groups of the NH₂—ZnO films to form imines and subsequent dispersion of the protective, outer layer of the NH₂—ZnO nanoparticles, which may render the NH₂—ZnO nanoparticles more susceptible to photobleaching.

### Example 5

The NH₂—ZnO films fabricated as described in Example 1 were evaluated for their potential for use as FRET donors by attaching a FRET acceptor (e.g., an organic, fluorescent dye) directly to the NH₂—ZnO film,

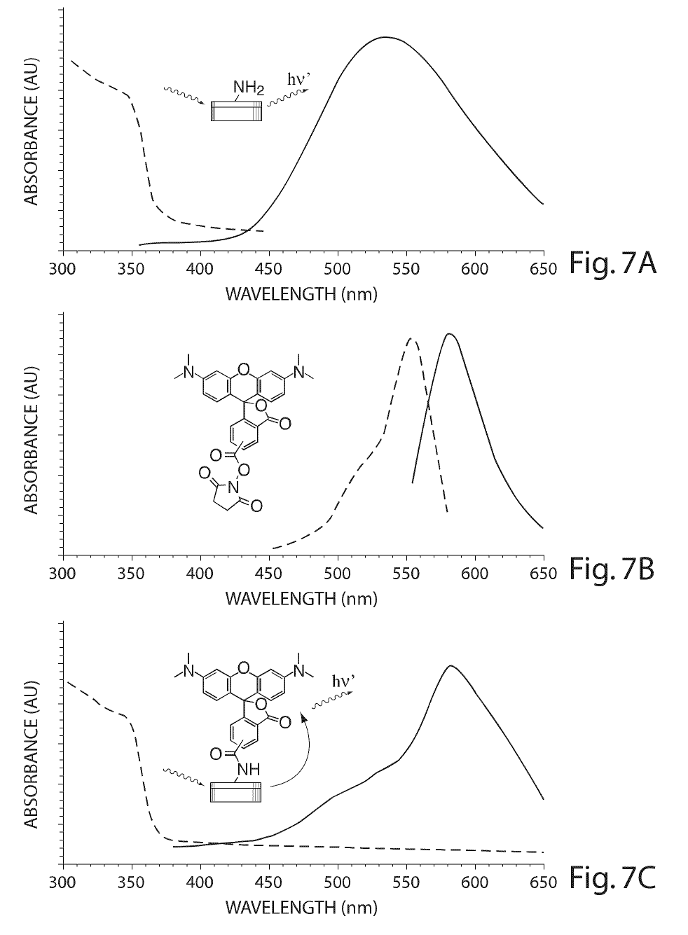

A NH₂—ZnO film was treated with a succinimidyl ester-activated tetramethylrhodamine (TMR) dye. FIG. 7A shows the absorption spectra (dotted line). and emission spectra (solid line) for a NH₂—ZnO film, excited at 345 nm. FIG. 7B shows the absorption spectra (dotted line) and emission spectra (solid line) for a TMR succinimidyl ester dye, excited at 545 nm. The TMR was selected for the broad spectral overlap between the emission spectrum of NH₂—ZnO film and the absorption spectrum of the TMR dye.

Fluorescamine was added to verify that substantially all the amino groups were functionalized with TMR molecules. Due to the low concentration of the surface NH₂ groups, absorption from grafted TMR groups could not be observed (FIG. 7C). However, as shown in FIG. 7C, direct excitation of the ZnO film (at 345 nm) resulted in an emission peak at 580 nm, which was attributed to emission from the TMR dye, rather than an emission peak associated with the ZnO film, expected to occur at 537 nm. These observations indicated that the excitation energy was transferred from the luminescent ZnO film to the TMR dye grafted on its surface.

### Example 6

The luminescent, amine-functionalized ZnO (NH₂—ZnO) films fabricated as described in Example 1 were further functionalized for use in a biological assay. A NH₂—ZnO film was functionalized with a biological binding partner that may selectively bind a target analyte. In this example, the NH₂—ZnO film was functionalized with a biotin moiety, which may selectively bind an avidin moiety or, alternatively, an avidin-biotin assembly. As shown schematically in FIG. 8A, the surface of a 1 NH₂—ZnO film was functionalized with N-hydroxysuccinimide-biotin (NHS-biotin) by immersion of the film in 10 mM of borate buffer containing 0.01 mg/mL NHS-biotin, as described at 4° C. for 6 hours. The film was then removed from the solution, and rinsed twice with deionized water. The procedure was repeated three times to afford the biotin-functionalized film (biotin-ZnO film). Due to the short half-life of NHS-biotin, the immersion was repeated three times, with freshly prepared NHS-biotin solution used for each immersion to increase the degree of biotin functionalization. Fluorescamine was added to evaluate the degree of biotin functionalization, and, upon observation of the luminescence intensity of the film, it was determined that 70% of the amino groups were converted to biotin. As a control experiment, a ZnO film was immersed in borate buffer without NHS-biotin and the luminescence intensity was compared to that of a biotinylated ZnO film. The luminescence intensity of the biotinylated ZnO film was found to be 50% lower than the un-functionalized ZnO film.

### Example 7

The biotin-ZnO film (Example 6) was then employed in as a biological sensor, using fluorescence resonance energy transfer (FRET) as the mechanism for signal transduction from the biotin-ZnO film (the FRET donor) to an organic, fluorescent dye (the FRET acceptor).

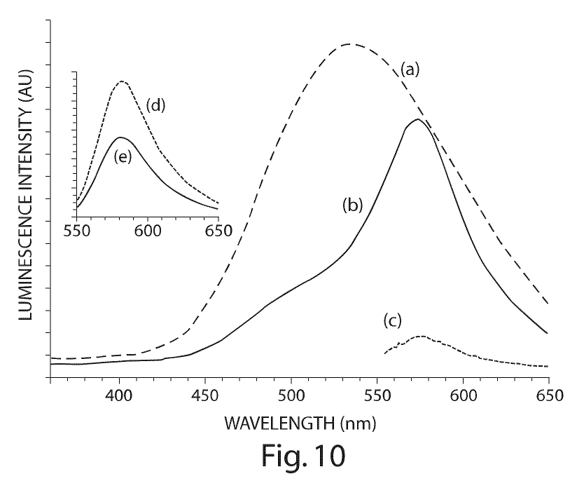

FIG. 8B shows, schematically, subsequent assembly of a TMR-substituted biotin/avidin/biotin-ZnO film structure for fluorescence resonance energy transfer (FRET). The biotin-ZnO film was first immersed in a solution of avidin, followed by subsequent immersion in a solution of TMR-tagged biotin to form the desired assembly. FIG. 10A shows the emission spectrum of biotin-ZnO film without the bound TMR dye, excited at the excitation wavelength of the ZnO film (345 nm). The emission spectrum of the TMR-biotin/avidin/biotin-ZnO film structure assembly shown in FIG. 10B, displayed reduced luminescence from the ZnO film and enhanced luminescence from the TMR dye when excited at 345 nm. This indicated, the occurrence of FRET between the ZnO layer and a tetramethylrhodamine dye bound to the ZnO layer through biotin-avidin-biotin assembly, shown schematically in FIG. 9.

Additionally, the emission intensity from the TMR dye due to FRET from the ZnO film was significantly greater than the emission intensity from the TMR dye upon direct excitation of the dye at 545 nm (FIG. 10C), illustrating the light-harvesting ability of the ZnO film. In contrast, the emission intensity from the TMR-biotin molecule in solution upon excitation of the dye at 545 nm (FIG. 10E) was about 60% greater than the emission intensity upon excitation at 345 nm (FIG. 10D). This may illustrate the ability of luminescent ZnO films to act as a strong, light harvesting tool for FRET.

While several embodiments of the invention have been described and illustrated herein, those of ordinary skill in the art will readily envision a variety of other means and structures for performing the functions and/or obtaining the results or advantages described herein, and each of such variations, modifications and improvements is deemed to be within the scope of the present invention. More generally, those skilled in the art would readily appreciate that all parameters, materials, reaction conditions, and configurations described herein are meant to be exemplary and that actual parameters, materials, reaction conditions, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the claims (as well as in the specification above), all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B” can refer, in one embodiment to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood, unless otherwise indicated, to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements that the phrase “at least one” refers to, whether related or unrelated to those elements specifically identified. Thus, as a non-limiting example, “at least one of A and B” (or, equivalently, “at least one of A or B,” or, equivalently ““at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

All references cited herein, including patents and published applications, are incorporated herein by reference. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

What is claimed:

1. A method for formation of a luminescent metal oxide nanoparticle thin film, comprising:
forming a layer comprising a luminescent metal oxide nanoparticle layer on a surface of a substrate; and
heating the substrate at a temperature of no more than 150° C. for a period of time sufficient to anneal the luminescent metal oxide nanoparticle layer to the surface,
wherein, prior to heating, the luminescent metal oxide nanoparticle layer has a first emission under a particular set of excitation conditions and, upon heating, the luminescent metal oxide nanoparticle layer has a second emission under the particular set of excitation conditions having at least 80% of the intensity of the first emission.

2. A method as in claim 1, wherein, prior to heating, the luminescent metal oxide nanoparticle layer has a first emission under a particular set of excitation conditions and, upon heating, the luminescent metal oxide nanoparticle layer has a second emission under the particular set of excitation conditions having at least 90% of the intensity of the first emission.

3. A method as in claim 1, wherein the luminescent metal oxide nanoparticle layer comprises ZnO nanoparticles.

4. A method as in claim 1, wherein the luminescent metal oxide nanoparticle layer comprises a binding partner selected to preferentially bind a target analyte.

5. A method as in claim 1, wherein the target analyte is a biological or chemical analyte.

6. A method as in claim 1, wherein the luminescent metal oxide nanoparticle layer comprises a binding partner selected to preferentially bind a target analyte.

7. A method as in claim 1, wherein the luminescent metal oxide nanoparticle layer comprises a functional group selected from among amine, carboxylic acid, phosphate, hydroxyl, and thiol.

8. A method as in claim 1, wherein the functional group is an amine.

9. A method as in claim 1, wherein the functional group is a carboxylic acid.

10. A method as in claim 1, wherein the forming comprises spin-casting or drop-casting the layer from a solution comprising luminescent metal oxide nanoparticles.

11. A method as in claim 1, wherein the forming comprises spin-casting the layer from a solution comprising luminescent metal oxide nanoparticles.

12. A method as in claim 1, comprising heating the substrate at a temperature of no more than 130° C. for a period of time sufficient to anneal the luminescent metal oxide nanoparticle layer to the surface.

13. A method as in claim 1, comprising heating the substrate at a temperature of no more than 110° C. for a period of time sufficient to anneal the luminescent metal oxide nanoparticle layer to the surface.

14. A method as in claim 1, wherein, upon heating, the luminescent metal oxide nanoparticle layer forms a covalent bond to the surface.

15. A method of binding an analyte, comprising:
exposing a luminescent metal oxide nanoparticle layer to a sample suspected of containing an analyte and, if the analyte is present, allowing the analyte to become immobilized with respect to the luminescent metal oxide nanoparticle layer via interaction between the analyte and the luminescent metal oxide nanoparticle layer.

16. A method as in claim 15, wherein the luminescent metal oxide nanoparticle layer comprises ZnO nanoparticles.

17. A method as in claim 15, wherein the interaction between the analyte and the luminescent metal oxide nanoparticle layer comprises binding between two biological molecules.

18. A method as in claim 15, wherein the interaction between the analyte and the luminescent metal oxide nanoparticle layer comprises forming a covalent bond.

19. A method as in claim 15, wherein the luminescent metal oxide nanoparticle layer comprises a binding partner selected to preferentially bind the analyte.

20. A method as in claim 15, wherein the binding partner is selected from among amine, carboxylic acid, phosphate, hydroxyl, and thiol.

21. A method as in claim 20, wherein the binding partner is an amine.

22. A method as in claim 20, wherein the binding partner is a carboxylic acid.

23. An article as in claim 15, wherein the binding partner comprises a biological molecule.

24. A method as in claim 15, wherein the binding partner comprises biotin.

25. A method as in claim 15, wherein the analyte comprises a fluorophore.

26. A method as in claim 25, wherein the fluorophore comprises a fluorescent dye.

27. A method as in claim 25, further comprising:
exposing the luminescent metal oxide nanoparticle layer to the sample suspected of containing the analyte, wherein the luminescent metal oxide nanoparticle layer is a fluorescence resonance energy transfer donor and the fluorophore is a fluorescence resonance energy transfer acceptor;
exposing the luminescent metal oxide nanoparticle layer to a source of energy to form a luminescent metal oxide nanoparticle excitation energy;
in the event that the analyte is present, allowing the excitation energy to transfer to the fluorophore, causing an emission from the fluorophore; and
determining the analyte via determination of the emission.

28. An particle for determination of a target analyte, comprising:
a substrate and a layer comprising luminescent metal oxide nanoparticles formed on and adhered to a surface of the substrate, wherein the luminescent metal oxide nanoparticles comprise a binding partner selected to preferentially bind the target analyte.

29. An article as in claim 28, wherein the luminescent metal oxide nanoparticle comprises ZnO.

30. An article as in claim 28, wherein the binding partner is selected from among amine, carboxylic acid, phosphate, hydroxyl, and thiol.

31. A method as in claim 30, wherein the binding partner is an amine.

32. A method as in claim 30, wherein the binding partner is a carboxylic acid.

33. An article as in claim 28, wherein the binding partner comprises a biological molecule.

34. An article as in claim 28, wherein the binding partner comprises biotin.

35. An article as in claim 28, wherein the target analyte is a biological or chemical analyte.

36. An article as in claim 28, wherein the target analyte comprises a fluorophore.

37. An article as in claim 28, wherein the fluorophore comprises a fluorescent dye.

38. An article as in claim 28, wherein the luminescent metal oxide nanoparticles are adhered to the surface of the substrate via covalent bonds.

39. An article as in claim 28, wherein the target analyte is bound to the luminescent metal oxide nanoparticle layer via binding between two biological molecules.

40. An article as in claim 28, wherein the target analyte is bound to the luminescent metal oxide nanoparticle layer via formation of a bond.

41. An article as in claim 40, wherein the bond is a covalent, ionic, hydrogen, or dative bond.

42. A fluorescence resonance energy transfer donor, comprising:
a luminescent metal oxide nanoparticle comprising a binding partner selected to preferentially bind an analyte, wherein the luminescent metal oxide nanoparticle is a fluorescence resonance energy transfer donor and the analyte is a fluorescence resonance energy transfer acceptor.

43. A fluorescence resonance energy transfer donor as in claim 42, further comprising a substrate and a layer comprising the luminescent metal oxide nanoparticles formed on and adhered to a surface of the substrate.

44. A fluorescence resonance energy transfer donor as in claim 42, wherein the luminescent metal oxide nanoparticle comprises ZnO.

45. A fluorescence resonance energy transfer donor as in claim 42, wherein the binding partner is selected from among amine, carboxylic acid, phosphate, hydroxyl, and thiol.

46. A fluorescence resonance energy transfer donor as in claim 42, wherein the binding partner is an amine.

47. A fluorescence resonance energy transfer donor as in claim 42, wherein the binding partner is a carboxylic acid.

48. A fluorescence resonance energy transfer donor as in claim 42, wherein the binding partner comprises a biological molecule.

49. A fluorescence resonance energy transfer donor as in claim 42, wherein the binding partner comprises biotin.

50. A fluorescence resonance energy transfer donor as in claim 42, wherein the target analyte is a biological or chemical analyte.

51. A fluorescence resonance energy transfer donor as in claim 42, wherein the target analyte comprises a fluorophore.

52. A fluorescence resonance energy transfer donor as in claim D7, wherein the fluorophore comprises a fluorescent dye.

53. A fluorescence resonance energy transfer donor as in claim 42, wherein the luminescent metal oxide nanoparticles are adhered to the surface of the substrate via covalent bonds.

54. A fluorescence resonance energy transfer donor as in claim 42, wherein the target analyte is bound to the luminescent metal oxide nanoparticle layer via binding between two biological molecules.

55. A fluorescence resonance energy transfer donor as in claim 42, wherein the target analyte is bound to the luminescent metal oxide nanoparticle layer via formation of a bond.

56. A fluorescence resonance energy transfer donor as in claim 55, wherein the bond is a covalent, ionic, hydrogen, or dative bond.
